(19) United States
(12) Patent Application Publication
(10) Pub. No.: US 2020/0397332 A1
(43) Pub. Date: Dec. 24, 2020

(54) NON-INVASIVE METHOD AND DEVICE FOR DETERMINING DRUG CONCENTRATIONS IN BLOOD

(71) Applicant: Yevgeniy Postrekhin, Orlando, FL (US)

(72) Inventor: Yevgeniy Postrekhin, Orlando, FL (US)

(21) Appl. No.: 16/448,595

(22) Filed: Jun. 21, 2019

(51) Int. Cl. A61B 5/05; A61B 5/145; A61B 5/00

(52) U.S. Cl. CPC A61B 5/05; A61B 5/14546; A61B 17/132; A61B 2562/0223; A61B 5/4845

(57) ABSTRACT

The present invention relates generally to method and device for the detection of drug levels in blood flow using the application of magnetic fields for the detection of small electronic currents. The method of the present invention includes the electromagnetic measurements that are based on the readings of the sensitivity of a giant magnetoresistance sensor which senses the magnetic field induced by the ions of the circulating drug in arterial blood flow. The device includes a pressure cuff, a magnetic sensor, a permanent magnet, data acquisition box, and a graphical user interface to control input data and read the current drug concentration in the arterial blood flow.

### FIELD OF THE INVENTION

The present invention relates generally to non-invasive method and device for the detection of drug levels in blood flow using the application of magnetic fields for the detection of small electrical currents. The method of the present invention includes the electromagnetic measurements that are based on the readings of the sensitivity of a giant magnetoresistance sensor which senses the magnetic field induced by the ions of the circulating drug in arterial blood flow. The device includes a pressure cuff, a magnetic sensor, a permanent magnet, data acquisition box, and a graphical user interface to control input data and read the current drug concentration in the arterial blood flow.

### BACKGROUND OF THE INVENTION

The proposed invention provides an alternate non-invasive method and portable device to analyze drugs in the blood stream using a giant magnetoresistance sensor. This method and apparatus detect the absolute value of drugs in blood in real time based on measured magnetic sensor signals and may also include the use of a mathematical approach to recognize the concentration of the drug over time.

Drug concentration in blood can be determined by using non-invasive spectrophotometry measurements, namely, pulse oximetry measurements, wherein a pulsatile component of the optical signal obtained from blood perfused tissue for determining the arterial blood oxygen saturation. The other chemical elements can be determined by the same technique. This is measured as a time dependent component of the total light reflection or transmission signal obtained from tissue. The major drawback of this technique is low signal-to-noise ratio that is insufficient for providing a pulsatile signal suitable for accurate measurements. U.S. Pat. No. 4,883,055 discloses a method and device for artificially inducing blood pulse for use with a pulse oximeter. A cuff is wrapped around a finger having artery flow upstream from a testing site. Squeezing the cuff around a finger with synchronized pulse and normal blood pulse, oxygen saturation is determined based on spectrophotometric non-invasive measurements in the arterial blood.

U.S. Pat. No. 6,400,972 is based on dramatically changing the light response characteristics of the blood perfused medium, when causing blood flow cessation. The authors of this patent claim that signal-to-noise ratio dramatically increased. In this device, an over-systolic pressure is applied to a patient's blood perfused fleshy tissue. The pressure causes a state of temporary blood flow cessation within a time period insufficient for irreversible changes in the fleshy tissue. Release of the over systolic pressure causes a state of transitional blood flow terminating with the normal blood flow. At least two sessions of measurement, separated in time, are executed and at least one of these sessions is selected within the time period including the state of temporary blood flow cessation and the state of transitional blood flow. Optical non-invasive measurement of the concentration of at least one blood constituent are successively performed at these at least two sessions, and respective values of the concentration are obtained.

Another non-invasive method for determining drug concentration in blood including alcohol is the method utilizing sensors that can analyze exhaled breath components (U.S. Pat. Nos. 5,971,937, 6,010,459). In U.S. Pat. No. 5,971,937 the exhaled air is sampled for measured carbon dioxide concentration value which is proportional the blood alcohol concentration value. U.S. Pat. No. 6,010,459 describes the method and apparatus for the measurements of components of exhaled breath in humans. This method is non-invasive but utilizes stationary equipment as gas chromatography and mass spectrometry.

U.S. Pat. No. 8,180,427 describes an apparatus and method for non-invasively sensing pulse rate and blood anomalies. The disclosed invention provides an apparatus and method for non-invasively sensing pulse rate and blood flow anomalies using a localized, uni-directional, and constant magnetic field. The apparatus comprises a magnetic source for producing the magnetic field, a signal acquisition module with a magnetic sensor for detecting the modulations of the magnetic field caused by the blood flow; and a signal processing module for processing the acquired signals so as to produce data of pulse rate and blood flow anomalies. The method senses pulse rate and blood flow anomalies by providing a localized, uni-directional, and constant magnetic field in proximity to a blood vessel; detecting the variations of the magnetic field caused by the flow of pulsatile blood within the blood vessel; and processing the signals of the detected variations so as to monitor the blood flow.

The above patents give non-invasive methods for determining drug concentrations in the flow of blood through blood vessels and exhaled breath components. The described optical methods require improved signal-noise ratio and the exhaled methods are useful for particular drugs such as alcohol or other drugs that can be detected in a subject's exhalation under controlled conditions such as in a hospital.

### SUMMARY OF THE INVENTION

The present invention relates generally to method and device for the detection of drug levels in blood flow using the application of magnetic fields for the detection of small electronic currents. During the metabolism of a drug in the blood stream, various blood circulating cations and anions create a cumulative small electrical current difference which induces a magnetic field around the blood vessel. The method of the present invention includes the use of highly sensitive electromagnetic measurements that are based on the readings of a giant magnetoresistance sensor which senses the magnetic field induced by the ions of the circulating drug in arterial blood flow. The proposed non-invasive method also includes the mathematical approximation for time dependent concentration of drug in the arterial blood flow and the usage of known drug data, namely, the maximum absorption drug concentration of the blood flow over time with a certain initial dose.

The device includes a pressure cuff, a magnetic sensor, a permanent magnet, data acquisition box, and a graphical user interface to control input data and read the current drug concentration in the arterial blood flow. The magnetic sensor signal difference is detected at the moment before squeezing a cuff and after squeezing the cuff to provide an indication of the drug concentration in the arterial blood flow. Using a database for maximum drug absorption in blood at a certain initial dose for a drug and the time dependent magnetic signal from the magnetic sensor, the absolute value of the drug concentration in the arterial blood stream is determined.

The arterial blood system is responsible for the transport of the blood which transmits oxygen, nutrients and other solutes at cellular level of the tissues. These other solutes include ions such as sodium Na+, potassium K+, magnesium Mg+, calcium Ca+, chloride Cl−, PO₄²⁻, HCO₃⁻, OH−. According to basic principles, the anions and cations are trying to reach equilibrium, such that the total number of positive charges are balanced with the total number of the negative charges. The equilibrium is true for a static system. For the dynamic picture, the anions and cations are close to equilibrium but do not reach it. In the arterial blood system, there is an unbalance between positive and negative charges. In the medical field, there are terms such as anion gap, the difference between major cation Na+ and major anions Cl− and NCO₃⁻, a strong ion difference, as an unmeasured difference between cations and anions, and so on. (Editorials, What exactly is the strong ion gap, and does anybody care? Clinical Care and Resuscitation 2004; v6, pp 155-166). The total flow of ions in blood creates an electrical current that produces a magnetic field around the blood vessel. Using giant magnetoresistance (GM) sensor it is shown how the arterial blood flow, reacts to a variety of internal factors. The internal factors may include drugs such as alcohol, caffeine and other medications. A primary reason for an imbalance of the positive and negative ions, which create the electric current fluctuations, are metabolic events happening inside the arterial blood flow. The metabolic events to consider include the following electrochemical reactions: H₂O=H++OH−, C₃H₅O₃ (lactate)+H++3O₂=3H₂O+3CO₂, OH−+CO₂=HCO₃−, C₃H₅O₃−+302=2H₂O+2CO₂+HCO3− and other reactions related to the metabolic acidosis of the drugs itself.

Blood is a concentrated suspension of several formed cellular elements as red blood cells, white blood cells and platelets dissolved in an aqueous ionic solution, the plasma. This solution is composed of about 93% water and about 7% particles, namely, electrolytes, organic molecules and numerous proteins. The moving electrolytes are the source of the magnetic field. The blood itself is nonmagnetic. The magnetic susceptibility of the blood is χ=−4.6 10⁻⁶. M. E. Cana, R. Casta\tilde{ } neda-Priego, A. Barreraa, J. C. Estradaa, P. Knautha, and M. A. Sosa Magnetisation of red blood cells: a Brownian Dynamics Simulation, Revista Mexicana de Fisica 58 (2012) 391-396. Blood in the Earth magnetic field is approximately μμ₀H=10⁻⁵ T where μ is magnetic permeability of air that is close to 1 and μ₀ is the permeability of a vacuum which is approximately 4π10⁻⁷ H/m and H is magnetic field. The magnetic induction close to the blood vessel will be B=μμ₀(1+χ)H=μμ₀H+μμ₀χH. μμ₀χH is the contribution to the magnetic field related to the magnetic properties of the blood and it is approximately 10⁻¹¹ T. This value is beyond the sensitivity of the GM sensor alone.

The blood vessel of a patient can be considered to be the equivalent of a rigid tube with length L and area S where the inside of the blood vessel is the arterial blood The arterial blood flow moves with positive + and negative − ions species with velocity vᵢ, volume concentration of each species nᵢ and charge of every species +q or −q, with mass mᵢ. The external magnetic field B and electric field E can be included in this model. The electrical current density for each species will be J=qnᵢvᵢ and the total current density is J=Σᵢ qnᵢ vᵢ

Hence the current I=(Σᵢqnᵢvᵢ/S

The behavior of the arterial blood flow in the electromagnetic field can be described by a simplified model of the electromagnetic plasma with several types of ions. The blood vessel may also be considered similar to an element of a linear wire. The magnetic induction B of the current I going through linear wire is

B=(μμ₀/4π)(2I/r)

Where μ is magnetic permeability of air (around 1), μ₀ is magnetic permeability of the vacuum (4π×10⁻⁷ H/m); r is distance from the surface of the wire. In fact, vᵢ (t) is directly related to the magnetic field outside the blood vessel as nivi (t)˜B(t).

The total number of ions inside the arterial blood flow creating the electrical current is estimated using the fact that 1 mol of matter contains ˜10²³ units, parts, or particles. (Avogadro number 6.02×10²³) and taking typical concentrations of ions inside the arterial blood flow. Na+ is 139.8 mmol/liter, K+ is 3.96, Ca+ is 2.38, Mg+ is 0.83 and Cl− is 103.0. The average velocity for each ion as v=0.01 m/s and I˜(qvS)Σnᵢ, q˜10⁻¹⁹ C (charge of electron 1.6×10⁻¹⁹), calculating Σni˜40×10²³ m⁻³, S=10⁻⁶ m². The total electrical current with positive charges will be around 10 mA. The magnetic field is estimated using B=(μμ₀/4π)(2I/r), taking I=10 mA, r=10⁻³ m, μ=1 μ₀=4π10⁻⁷H/m. The magnetic induction will be around B=10⁻⁶ T which is equal to 10⁻² Gauss.

In order to measure the magnetic field of the arterial blood flow, the magnetic sensor must have sensitivity of approximately 10⁻² Gauss or greater. A Giant magnetoresistance (GM) magnetic sensor such as model AAH-002 (NVE Corp.) has sufficient sensitivity to measure the magnetic field produced by the electric current from the arterial blood flow. The sensitivity of the Giant magnetoresistance sensor is approximately 3 Gauss per 1 volt. The GM sensor is placed above one of primary wrist arteries with an adjacent small permanent magnet such as a pellet magnet with diameter 0.01 m and a field around 10 Gauss to work with this sensor in a non-hysteresis linear area. 10⁻² Gauss is equal to approximately 3 mV measured by the GM sensor to provide sensitivity in the range of the magnetic field from about 20 Gauss to 10⁻⁴ Gauss. The GM signal is also a function of the cations and anions at concentration Σn which are responsible for the electrical current and the GM sensor signal will change in response to external or internal factors acting on the blood flow.

A drug within the blood stream changes the balance of ions and the changes of concentration of ions. The concentrations of ions is directly dependent on the drug concentration in the blood over time. For instance, the concentration of caffeine in the arterial blood stream has a maximum level at about 30 min to 1 hour 30 minutes after ingesting one cup of coffee (100 mg caffeine). The caffeine level then declines slowly to zero after about 4 hours. The time dependent concentration of alcohol in blood has a maximum concentration at about 1 to 1 hour 30 minutes after ingestion and slowly decreases over time. After about 12 hours there is no sign of alcohol in blood.

The time dependent concentration of any drug in blood has similar features. A sharp increase beginning a certain time after ingestion, a maximum level at a certain time after ingestion, and a decrease to zero level. One possible way to describe the behavior of relative changing drug concentration over time is to introduce a mathematical relation between the drug concentration N and time t as dN/N(t)=(t/tmax)exp(−0.5(t/tmax)²). tmax is the time of the maximum drug concentration.

### BRIEF DESCRIPTION OF THE DRAWINGS

FIG. 1 is a diagrammatical view of a blood vessel;

FIG. 2 is illustrative of the setup for the GM sensor on a patient;

FIG. 3 is a block-diagram of the device to measure the drug concentration in the arterial blood flow;

FIG. 4 is illustrative of the GM signal vs time during a 10 second sample period;

FIG. 5 is illustrative of the GM signal vs time with palm muscle contractions;

FIG. 6 is illustrative of the GM magnetic signal vs time at different cuff pressure on the arm;

FIG. 7 shows the signal Vs vs time before ingesting the drug caffeine;

FIG. 8 shows the signal Vs vs time after 1 hour of ingesting one cup of coffee;

FIG. 9 shows the dependence of the relative changing ions inside the arterial blood stream vs time after drinking one cup of coffee;

FIG. 10 shows the signal Vs vs time before ingesting acetaminophen;

FIG. 11 shows the signal Vs vs time 1 hour after the ingestion of acetaminophen;

FIG. 12 shows the dependence of the relative changing ions inside the arterial blood stream vs time after ingesting acetaminophen; and

FIG. 13 is illustrative of Tmax as a graphical solution and the time dependent drug concentration.

### DETAILED DESCRIPTION

The method of determining the concentration of a drug in the arterial blood flow based on the charged components of arterial blood flow includes the step of obtaining electromagnetic measurements. These measurements enable the detection of the presence of various cations and anions moving through the blood. The cations and anions induce a small electrical current which is detected by the present system. The electromagnetic measurements are based on the high sensitivity of a giant magnetoresistance sensor which can sense the magnetic field induced by the slightly charged ions in arterial blood flow. The proposed non-invasive method also includes the reference to an approximation for time dependent concentration of a drug in the arterial blood flow and the usage of the known data for a particular drug, namely, the drug concentration of the blood flow at certain times based on initial drug ingestion and dosage to determine the optimal time for obtaining subsequent data collection.

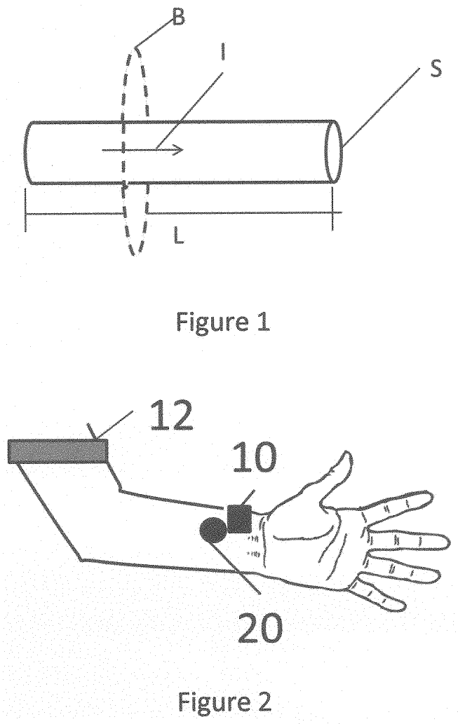

The concentration of the drug of interest over time is dependent on the concentration of the drug in the arterial blood flow. This is detected by monitoring the electrical current of the ions in the arterial blood flow changing over time as shown diagrammatically in FIG. 1. FIG. 1 is illustrative that the blood vessel as a rigid tube with length L and area S where the arterial blood is inside the blood vessel. The arterial flow consists of the flow of blood moving with positive + and negative − ions species with velocity vi, volume concentration of each species ni and the charge of every species +q or −q, with mass mi. The current density for each ion J=qnivi, and current I=qnivixS. The total current is ΣqnivixS. The magnetic induction B of the current I passing through a linear wire is B=(μμ0/4π)(2I/r) and nivi˜B.

The electrical setup described herein is built to amplify the signal from the GM sensor. In the preferred configuration shown in FIG. 2, The GM sensor 10 may be as an example model AAH-002 sensor from NVE Corp. The GM sensor 10 is positioned along the arm of a patient close to a major blood vessel with oxygenated blood. The GM sensor is positioned near a conventional permanent magnet 20 and the signal from the GM sensor is amplified by conventional preamplifier or amplifier 30. The data from the GM sensor is collected through a data acquisition box 40 and is analyzed by the standard computer 50. An example of a data acquisition box is manufactured by National Instruments Inc. The LabView hardware and software in the computer are used to analyze the signal from the GM sensor 10. A conventional computer may be programmed with commercially available software such as LabView software to analyze and subsequently display the signals reviewed from the GM sensor. A standard pressure cuff 12 is located upstream of the GM sensor and is controlled manually or through the data acquisition box 40 by the computer 50. The preferred configuration of the GM sensor 10, the permanent magnet 20, and the pressure cuff 12 on the arm of a patient are shown in FIG. 2.

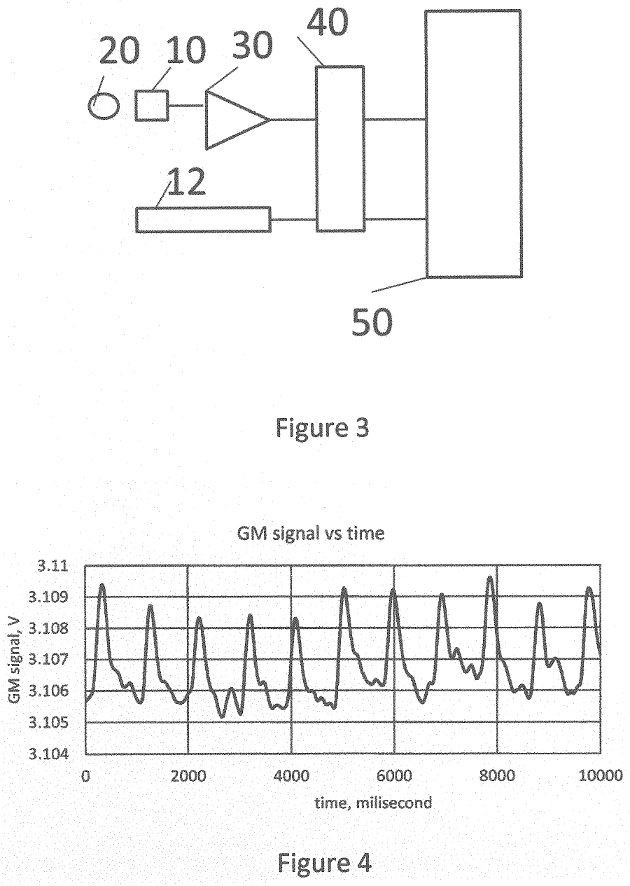

The flow diagram of the signals of the preferred form of the present invention is shown in FIG. 3. The detected signal passes from the GM sensor to the amplifier. The signal then passes from the amplifier 30 to the data acquisition box 40 and to the computer 50 for analysis. As shown, the pressure cuff 12 is controlled and monitored by the data acquisition box 40 and computer 50.

FIG. 4 is illustrative of the typical GM signal vs time during a 10 second sample. The pulsating shape is due to the heartbeat of the patient. The shape is also not perfect because of noise from the surrounding electrical insatiability including interference from the Earth magnetic field and the GM sensor 10 not being in a completely stable position on the arm of the patient.

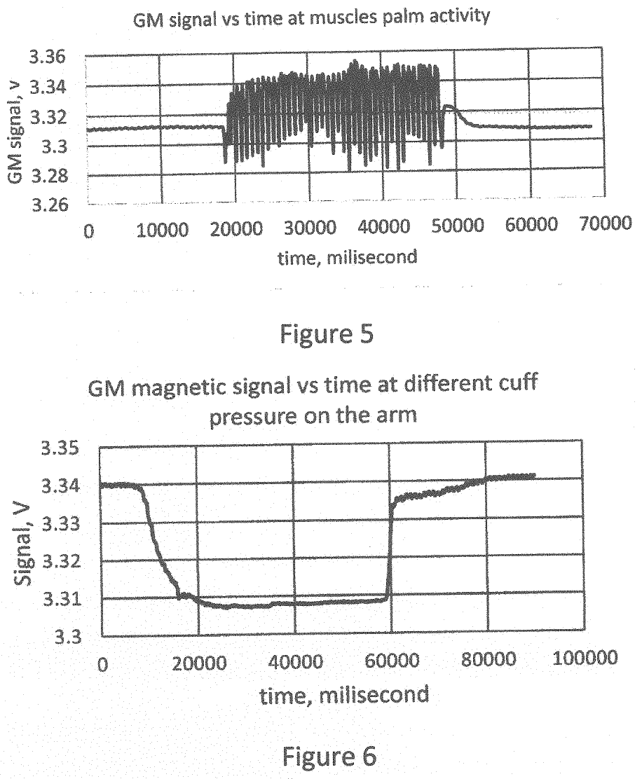

FIG. 5 is illustrative of the GM signal vs time showing muscle contractions of the palm of the patient. FIG. 5 presents the GM signal vs time showing the effect of muscle activity based on clenching and unclenching the hand of the patient every second over a 30 second time span. The big oscillations on the graph are due to the muscle movement close to the GM sensor 10. The average level of the signal is rising because the muscles require more energy as the muscle fatigues. The increasing energy requirements are satisfied by increasing the flow of blood and increasing the arterial blood flow to the muscles in the arm. This is confirmed by modeling the arterial blood flow and the changing electrical current detected by the GM sensor 10.

FIG. 6 is illustrative of the GM magnetic signal vs time at different pressures by adjusting the pressure applied by the pressure cuff 12 on the arm of the patient. Initially, the cuff pressure is zero and the GM signal is flat. After around 10 seconds, the cuff pressure is increased and after 20 seconds, the cuff pressure reaches approximately 120 mm Hg. Further increases of the cuff pressure up to 200 mm Hg does not change the GM signal. The GM signal is almost flat from 20 seconds to 60 seconds because the flow of blood through the blood vessel is stopped. When the cuff pressure was released quickly at about 60 seconds, the GM signal returned to the level similar to the initial measurement. At the pressure of 120 mm Hg, the arterial blood vessel is completely blocked and the flow of blood through the blood vessel is stopped. Further increases of the pressure in the pressure cuff does not change the GM signal because there is no flow of blood through the arterial blood vessel. At the pressure of 120 mm Hg or greater there is, no arterial blood flow and no electrical current. The difference of two levels in the beginning and around 60 seconds gives the absolute value of the magnetic field which is detected by the GM sensor. At time t=0 seconds, the mean value of the signal is Vs₀ and at the end of the event, when the cuff pressure is high and no blood flows through the arterial blood vessel, the signal is Vs. The difference (Vs₀−Vs)₀ represents the total ion current of the blood flow at the beginning time t=0. The concentration of the ions Σn=N detected in the arterial blood flow is affected by the drugs which have been ingested. Tracing the difference (Vs₀−Vs) before, during and after the drug ingestion, one can identify the level of the drug in the arterial blood flow over time. The difference (Vs₀−Vs)=(Vs₀−Vs)₀ before the ingestion of drug.

The signal before drug ingestion Vs₀ is proportional to the ion concentration N. So Vs₀=AN+B, where A and B are constants, A is a proportionality coefficient and B is the additional voltage which depends on the local magnetic environment (earth magnetic field H, artificial magnetic sources from power lines and so on. Furthermore, Vs=B because there is no blood flow through the artery when the cuff squeezes the arm, (Vs₀−Vs)₀=AN. After drug ingestion Vs₀=A(N+dN)+C and Vs=C because there is no blood flow through artery when the cuff squeezes the arm at a pressure greater than 120 mm Hg, and (Vs₀−Vs)=A(N+dN). Next, (Vs₀−Vs)=A(N+dN) is divided by Vs0=A(N+dN) and 1−(Vs₀−Vs)/(Vs₀−Vs)₀=dN/N.

Measuring Vs₀ and Vs and calculating 1−(Vs₀−Vs)/(Vs₀−Vs)₀ for multiple time periods gives the concentration of the cumulative ions over time. Because the location of the sensor on the arm is so sensitive, it is not practical to measure Vs continuously. It is desirable that the sensor and hand be maintained in a stable position for a long time and is difficult to monitor the signal in real time when the signal is monitored for hours.

The dependence 1−(Vs₀−Vs)/(Vs₀−Vs)₀=dN/N vs time shows how the relative concentration of the drug is changing over time where N is total sum of ions contributing to the detected ion current. The absolute value of the drug concentration as a function of time for different initial dosages is well known for many drugs. These data points can be saved in the computer and may be used as initial data to find the absolute value of the drug concentration in the arterial blood flow. Basic data for the detected drugs includes the initial dosages of the drug Din which depend on the maximum concentration Cmax in blood at a certain time Tmax. The basic features of the drug concentration time dependence are the increase in the beginning, to the maximum Cmax at Tmax and slowly decreasing to 0 concentration over a certain time. Such behavior can be fit by function dN/N(t)=(t/tmax)exp(−0.5(t/tmax)²). The Tmax can be identified from this dependence. The absolute value of the drug concentration is determined based on the measured signal Vs and time Tmax at which the concentration of drug is detected at a maximum value in the blood stream.

### Example 1

The signal Vs was measured at different times after ingestion of the caffeine to verify the time dependence of the drug concentration in blood. As example, it is presented Vs verses time before ingestion of the caffeine and after one hour after ingestion of the caffeine.

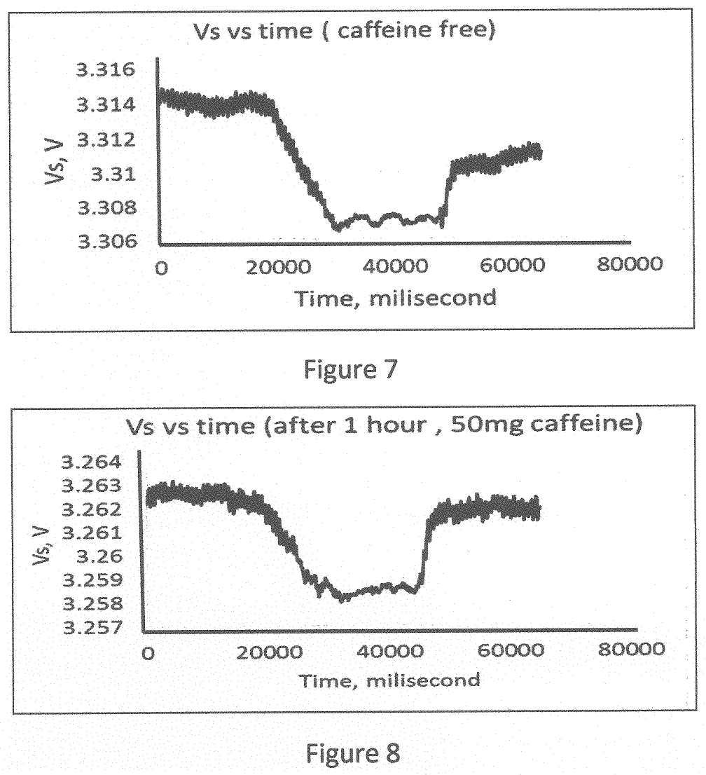

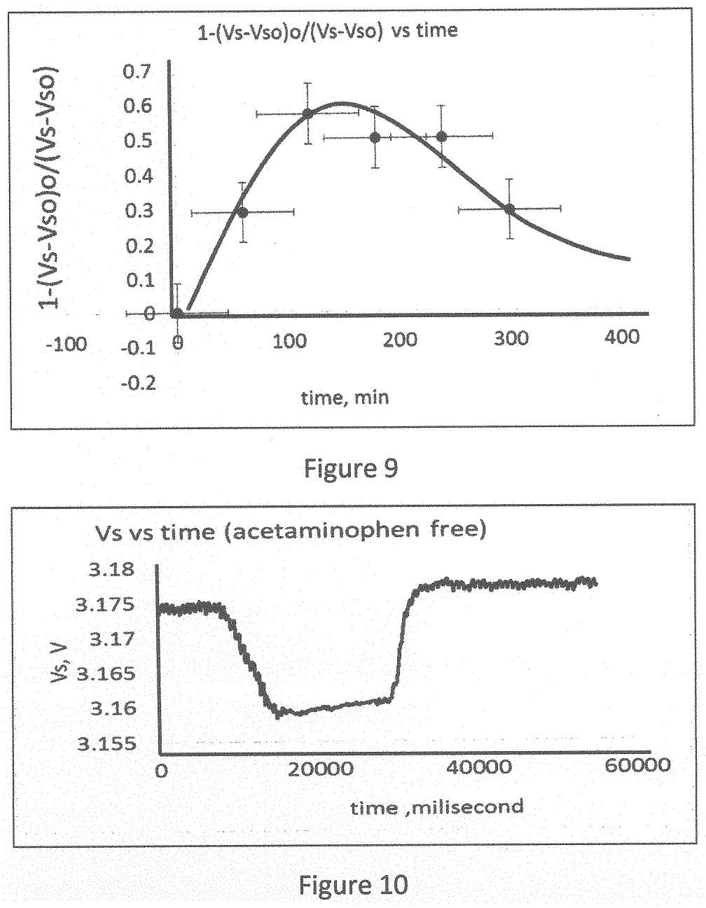

FIG. 7 shows the signal Vs vs time before using drug caffeine and showing the range for (Vs₀−Vs) of approximately 0.007 V. As shown in FIG. 7, the pressure from the pressure cuff 12 is approximately zero to about 20 seconds. The pressure is then increased from about 20 to 50 seconds and is greater than about 120 mmHg after about 50 seconds. FIG. 8 shows the signal Vs vs time about 1 hour after drinking one cup of coffee (˜50 mg of caffeine). The range for (Vs₀−Vs) is approximately 0.005 V. As shown in FIG. 8, the pressure from the pressure cuff 12 is approximately zero to about 20 seconds. The pressure is then increased from about 20 to 50 seconds and is greater than about 120 mmHg after about 50 seconds. FIG. 9 is illustrative of the relative change in ion charges inside the arterial blood stream vs time after drinking one cup of coffee (˜50 mg caffeine). The dependence 1−(Vs₀−Vs)/(Vs₀−Vs)₀=dN/N vs time is presented in FIG. 9 after drinking one cup of coffee (˜50 mg). FIG. 9 is illustrative that the relative concentration of caffeine is changing in time where N is total sum of ions contributing to the current due to the ions in the circulating arterial blood. The absolute value of the caffeine concentration as a function of time for different initial dosages is well known from various published scientific references. Such data is also available for caffeine, acetaminophen and other drugs including alcohol. For Example, the rate of absorption and relative bioavailability of caffeine administered in chewing gum versus capsules to normal healthy volunteers is published by Gary H. Kamimori, Chetan S. Karyekar, Ronald Otterstetter, Donna S. Cox, Thomas J. Balkin Gregory L. Belenky, Natalie D. Eddington in the International Journal of Pharmaceutics 234 (2002) 159-167 for caffeine and by L. Prescott for the kinetics and metabolism of paracetamol and phenacetin in Br. J. clin. Pharmac. (1980), 10, 291S-298S for acetaminophen. Basic data for these drugs includes the initial doses of the drug of interest Din which is dependent on the maximum concentration Cmax in blood at a certain time Tmax. The basic features of the drug concentration time dependence include the initial increase in detected ion changes at a certain time following ingestion of the drug. The detected ion changes increase to a maximum Cmax at Tmax and slowly decrease to 0 concentration after a certain time. Such behavior can be fit by function dN/N(t)=(t/tmax)exp(−0.5(t/tmax)²) and is shown by the solid line on FIG. 9. The Tmax can be identified from this graph, and it occurs at about ˜120 min.

The absolute value of the drug concentration is determined based on the measured signal Vs and time Tmax at which the concentration of drug is at a maximum value in the arterial blood. It is shown that Tmax is about 120 min from the Vs measurements and using the above described functions as an approximation. The initial dose of caffeine is Din˜50 mg of caffeine. The maximum drug concentration in the arterial blood stream is about 1 mg/L with at Din=50 mg and Tmax is about 120 min based on the GM signal measurements. Therefore, Cmax=1 mg/L as dN and Tmax as tin equation dN/N(t)=(t/tmax)exp(−0.5(t/tmax)²) to find the concentration of charges responsible for the electrical current in blood. Based on this experiment, the charges for the electrical current in the blood will be about N˜1.65 mg/L. The next step is to use equation dN/N(t)=(t/tmax)exp(−0.5(t/tmax)2) as a tool to identify the drug concentration in the blood at any sample time. dN=N(t/tmax)exp(−0.5(t/tmax)²); it will be dN=1.65(t/tmax)exp(−0.5(t/tmax)²) to determine the drug concentration at any time of interest.

### Example 2

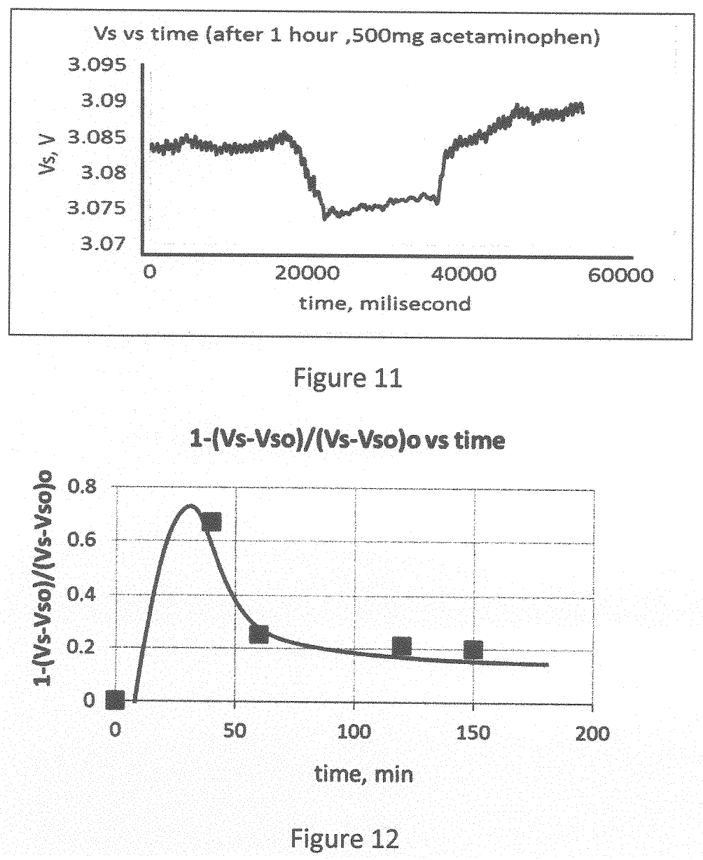

FIG. 10 is illustrative of Vs vs time immediately before the ingestion of acetaminophen and (Vs₀−Vs)₀ is determined to be approximately 0.015 V. As shown in FIG. 10, the pressure from the pressure cuff 12 is approximately zero to about 10 seconds. The pressure is then increased from about 10 to 30 seconds and is greater than about 120 mmHg after about 30 seconds. FIG. 11 shows Vs vs time after 1 hour following ingestion of the 500 mg dose of the acetaminophen and (Vs₀−Vs) is determined to be about 0.010 V. As shown in FIG. 11, the pressure from the pressure cuff 12 is approximately zero to about 10 seconds. The pressure is then increased from about 10 to 30 seconds and is greater than about 120 mmHg after about 30 seconds. FIG. 11 is illustrative of the relative change in charge of the ions inside the arterial blood stream vs time after ingesting 500 mg of acetaminophen. The dependence 1−(Vs₀−Vs)/(Vs₀−Vs)₀=dN/N vs time is presented in FIG. 12 after ingesting 500 mg of acetaminophen. This figure shows how the relative concentration of acetaminophen is changing in time where N is total sum of ions contributing to the ion currents detected in the atrial blood. The absolute value of the acetaminophen concentration as function of time for different initial dosages is well known. The basic features of the drug concentration time dependence are the initial increase in detected ion changes at a certain time following ingestion of the drug. The detected ion changes increase to a maximum Cmax at Tmax and slowly decrease to 0 concentration after a certain time. Such behavior can be fit by function dN/N(t)=(t/tmax)exp(−0.5(t/tmax)²)− as shown in the solid line on FIG. 12. The Tmax can be identified from this graph, and is detected as being approximately 30 min.

The initial dosage is Din is ˜500 mg of acetaminophen. The maximum drug concentration in arterial blood stream is 60 mg/L at Din=500 mg (˜60 mg/kg) and Tmax˜30 min from the measurements from the GM sensor. Placing Cmax=60 mg/L as dN and Tmax as t in equation dN/N(t)=(t/tmax)exp(−0.5(t/tmax)2) to find real concentration of charges responsible for electric current in blood. It will be N˜98.9 mg/L. The next step is to use equation dN/N(t)=(t/tmax)exp(−0.5(t/tmax)2) as a tool to identify the drug concentration in the blood at any time where dN=N(t/tmax)exp(−0.5(t/tmax)²); it will be determined that dN=98.9(t/tmax)exp(−0.5(t/tmax)²) for the time of interest.

The process for obtaining the measurements from the GM sensor include the step of taking the first measurement Vs₀. This initial reading establishes the baseline for background readings from the GM sensor while the blood is flowing through the blood vessel. Next, the pressure cuff is pressurized, and the second measurement is taken. This reading establishes the background reading for when the blood flow is stopped in the blood vessel. Vs. The next step is to calculate (Vs₀−Vs)₀.

The real absolute value of a drug can be determined based on the time at which the maximum absorption of the drug is observed in the blood vessel. This value is based on the maximum absorption drug concentration which is known from available databases for the drug of interest. This value is based on the mathematical model describing the time dependent drug concentration in the blood vessel. Once the drug of interest is ingested, further measurements are taken at a variety of times post ingestion of the drug. These measurements are taken with the cuff unpressurized and pressurized. For each pair of measurements, the calculation for (Vs₀−Vs) is made. Next, the calculation is made where 1−(Vs₀−Vs)/(Vs₀−Vs)₀ to approximate dN/N. Next, the approximation is calculated as dN/N=(t/tmax)exp(−0.5(t/tmax)²) to find Tmax. From the known drug data, the known data for the absolute maximum drug concentration Cmax in blood at the initial dosage is used. Next, the Tmax from the GM sensor measurements and the Cmax from known data is used to find absolute value N. Using ratio dN=N(t/tmax)exp(−0.5(t/tmax)²) the real absolute value of the drug is determined at a time of interest.

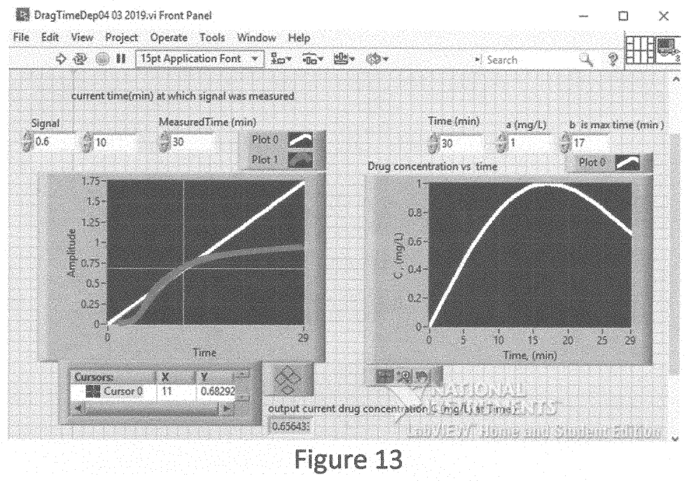

As shown in FIG. 13, the LabView software display is provided as a separate example, the left window shows two graphs giving tmax as a graphical solution. Using dN/N(t)=(t/tmax)exp(−0.5(t/tmax)2). The tmax can be identified from this dependence graphically. dN/N(t)=s is the signal from the measurements from the GM sensor. t=c is current time at which s signal measured. Tmax is an unknown value and tmax=x. The above equation will be s=(c/x)exp(−0.5(c/x)2), or sx/c=exp(−0.5(c/x)2). This equation can be solved graphically relative to x. The left window of FIG. 13 represents this graphical solution to this equation where the straight line between about 0-0 to 1.75-29 is the function sx/c and the curve line beginning at about 0-0 and ending about 0.9-29 is the function exp(−0.5(c/x)2). Intersection of this two lines gives x=tmax. This is tmax in our examples. The right graph gives the time dependent drug concentration for a specific drug at a specific drug dose. The steps for data acquisition and analysis in this example include first, opening the commercially available signal analysis software program such as the LabView application and measuring the response from the GM sensor. Second, the drug is ingested and after 10-20 mins (or whenever desired) measure the response from the GM sensor. Next determine the time at which the drug concentration will be at its maximum. Third, enter all data into the computer program. This data includes elapsed time after drug usage; maximum drug concentration at certain total drug usage from a data base and the time at which drug concentration is at maximum. Forth, at the current time following ingestion, the computer application will show the expected current drug concentration in the blood. The signal from the GM sensor is proportional to the ion concentration in the blood vessel. The concentration follows the metabolic reactions inside blood vessel to provide the exponential time dependent function. This fact allows the user to determine certain unknown parameters. In the present situation, this is tmax. The maximum time, tmax can be found at any time based on a single one-point measurement of the signal by the GM sensor as shown below. Using dN/N(t)=(t/tmax)exp(−0.5(t/tmax)2) The tmax can be identified from this dependence graphically. dN/N(t)=s is the signal from the measurements. t=c is current time at which s signal measured.

The present invention includes several suggestions and limitations to simplify the analysis of arterial blood flow and to determine the flow as the electrical current producing the magnetic field around the arterial blood flow in the selected vessel. In the above described method; the given model of the blood flow has only a few controllable parameters and constants. Despite the presence of limited variables, this model can predict features related to the ions in the arterial blood flow. For instance, if the electric current of the arterial blood flow increases or decreases certain amounts of the ions in the blood flow correspondingly increase or decrease. The reason for such changes includes the metabolism of drugs inside the blood flow. This model of the arterial blood flow as the electric current is applied to the larger blood vessels. The blood flow in microcirculation where the size of the vessels is hundreds of microns and smaller, can be described on the microcellular level. But processes such as metabolism, oxygen delivery, electro-diffusion through cell membrane and other functions have a definite effect on the whole blood system as well as the arterial blood flow. So, when something happens on the microcirculation blood level, the arterial blood flow will immediately react to these changes. It takes only a few seconds for the arterial blood flow to react to the muscle contractions or the internal factors such as drug metabolism and the use of the GM sensor for these fluctuations is currently not feasible.

The arterial blood flow of the selected artery is shown as the source of the electrical current which produces the magnetic field around arterial blood vessels. This electrical current is a result of the ions moving inside the arterial blood flow. The model of the arterial blood flow includes plasma with ions Na+, K+, Ca+Mg+, Cl−, PO₄− and organic ions which are dependent on the type of drugs inside the arterial blood flow. Using giant magnetoresistance sensor, the magnetic field of the arterial blood flow was measured. Changes in the absolute quantities of the ions inside blood can indicate the emotional stress, dehydration, muscle contractions, and drugs and can be measured by giant magnetoresistance sensor of the present invention.

It is anticipated that the system of the present invention may be modified and utilized to detect the peak and trough concentrations of the drug of interest to assist in providing the optimal dosing for the drug of interest. Additionally, the present invention may be used to detect the concentration of the drug of interest to ensure that the drug concentration is maintained within the desired therapeutic range for the drug. Furthermore, the present invention may be used to ensure that the drug of interest is not in a toxic or subtherapeutic range. It is also anticipated that the present invention may be used to classify patients as fast or slow metabolizers of certain drugs and may be used to allow the physician to increase or decrease the dosing of a drug of interest accordingly or to adjust the dosing based on the kidney or liver function of the patient which may be indicated by the altered presence of ions in the blood vessel.

What is claimed is:

1. A non-invasive method for determining the concentration of a drug in the blood vessel of a patient, comprising;
Providing a giant magnetoresistance sensor to detect the ion charge of blood flowing through a blood vessel of a patient; and
Adjusting the subsequent pressure applied to the blood vessel of a patient while obtaining signals of the ion charge in the blood vessel of the patient from the giant magnetoresistance sensor to determine magnetic field generated by the blood vessel in response to the concentration of the drug of interest in the blood vessel.

2. The method of claim 1 wherein a pressure cuff is configured to apply a varying pressure to the blood vessel of a patient to allow the giant magnetoresistance sensor to determine the ion charge prior to the administration of a drug of interest and at one or more times following the ingestion of the drug of interest.

3. The method of claim 2 wherein a computer is configured to control the application of varying pressure to the blood vessel of the patient by increasing or decreasing the pressure applied by the pressure cuff.

4. The method of claim 1 wherein the sensor is a giant magnetoresistance sensor to detect the effect of the magnetic field on the target tissue and a permanent magnet is positioned generally adjacent to the giant magnetoresistance sensor in a non-hysteresis linear area on the selected area of the patient.

5. The method of claim 1 wherein the signal from the giant magnetoresistance sensor is transmitted to an amplifier.

6. The method of claim 5 wherein the signal from the amplifier is transmitted from the amplifier to a computer for display and analysis using a signal analysis program.

7. The method of claim 1 wherein the giant magnetoresistance sensor detects the ion charge of blood flowing through the blood vessel of a patient at one or more times following the ingestion of a drug of interest.

8. The method of claim 1 wherein the giant magnetoresistance sensor detects the ion charge of blood flowing through the blood vessel of a patient prior to and following the ingestion of a drug of interest.

9. The method of claim 1 wherein the giant magnetoresistance sensor is positioned to sense the flow of charged ions through the blood vessel of a patient and the giant magnetoresistance sensor supplies signals relating to the change in the flow of ions through to blood vessel to a computer for analysis to determine the relative level of the drug of interest in the blood vessel.

10. The method of claim 1 wherein the giant magnetoresistance sensor is positioned to sense the flow of charged ions through the blood vessel of a patient and the giant magnetoresistance sensor supplies signals relating to the change in the flow of ions through to blood vessel to a computer for analysis to determine the maximum level of the drug of interest in the blood vessel at a certain time following ingestion of the drug.

11. A non-invasive method for determining the concentration of a drug in the blood vessel of a patient, comprising;
Providing a giant magnetoresistance sensor to detect the ion charge of blood flowing through a blood vessel of a patient;
Adjusting the subsequent pressure applied to the blood vessel of a patient while obtaining signals of the ion charge in the blood vessel of the patient from the giant magnetoresistance sensor to determine magnetic field generated by the blood vessel in response to the concentration of the drug of interest in the blood vessel; and
Supplying the signals from the giant magnetoresistance sensor to a computer to determine the level of drug present in the blood vessel of the patient at one or more selected times following the ingestion of the drug.

12. The method of claim 11 wherein a pressure cuff is configured to apply a varying pressure to the blood vessel of a patient to allow the giant magnetoresistance sensor to determine the ion charge prior to the administration of a drug of interest and at one or more times following the ingestion of the drug of interest.

13. The method of claim 12 wherein a computer is configured to control the application of varying pressure to the blood vessel of the patient by increasing or decreasing the pressure applied by the pressure cuff.

14. The method of claim 11 wherein the sensor is a giant magnetoresistance sensor to detect the effect of the magnetic field on the target tissue and a permanent magnet is positioned generally adjacent to the giant magnetoresistance sensor in a non-hysteresis linear area on the selected area of the patient.

15. The method of claim 11 wherein the signal from the giant magnetoresistance sensor is transmitted to an amplifier and the signal from the amplifier is transmitted from the amplifier to a computer for display and analysis using a signal analysis program.

16. The method of claim 15 wherein the signal analysis program is used to determine the absolute value of the drug in a blood vessel of a patient

17. A system for noninvasively detecting the concentration of a drug in a blood vessel comprising,
A giant magnetoresistance sensor for detecting the ion charge of blood flowing through a blood vessel of a patient,
A pressure cuff for adjusting the pressure applied to the blood vessel of a patient while obtaining signals of the ion charge in the blood vessel of the patient from the giant magnetoresistance sensor to determine magnetic field generated by the ion charge in the blood vessel in response to the concentration of the drug of interest in the blood vessel; and
A data acquisition box for receiving the signals from the sensor to determine the concentration of the drug of interest in the blood vessel.

18. The device of claim 17 further including an amplifier for magnifying the signal received from the giant magnetoresistance sensor.

19. The device of claim 17 further including control of the pressure cuff by the data acquisition box to vary the flow of blood through the blood vessel of the patient.

20. The device of claim 17 further including a permanent magnet that is positioned generally adjacent to the giant magnetoresistance sensor in a non-hysteresis linear area on a selected area of the patient.
